(19) United States
(12) Patent Application Publication
(10) Pub. No.: US 2025/0060355 A1
(43) Pub. Date: Feb. 20, 2025

(54) BIOCHIP FOR MEASURING CELL DENSITY OF TARGET BIO-PARTICLES

(71) Applicant: Southern Taiwan University of Science and Technology, Tainan City (TW)

(72) Inventors: I-Fang CHENG, Tainan City (TW); Jyun-Hong WANG, Tainan City (TW); I-Ling CHEN, Tainan City (TW); Pin-Ru KE, Tainan City (TW)

(21) Appl. No.: 18/802,464

(22) Filed: Aug. 13, 2024

Related U.S. Application Data
(60) Provisional application No. 63/519,685, filed on Aug. 15, 2023.

(51) Int. Cl. G01N 33/487; G01N 27/327

(52) U.S. Cl. CPC G01N 33/48735; G01N 27/3275

(57) ABSTRACT

A biochip for measuring a cell density of target bio-particles in a sample is provided. The biochip includes a spiral electrode pair and a concentric electrode array. The spiral electrode pair includes a first spiral electrode and a second spiral electrode that are parallel to each other and that are spaced apart from each other. Each of the first spiral electrode and the second spiral electrode is shaped in a spiral extending in one of clockwise and counterclockwise directions. The concentric electrode array includes a plurality of arcs that are concentric and extend in a circular direction around the spiral electrode pair.

### CROSS-REFERENCE TO RELATED APPLICATION

This application claims the benefit of U.S. Provisional Patent Application No. 63/519,685, filed on Aug. 15, 2023, and incorporated by reference herein in its entirety.

### FIELD

The disclosure relates to a biochip, and more particularly to a biochip for measuring a cell density of target bio-particles in a sample.

### BACKGROUND

Referring to FIG. 1, a conventional biochip for measuring a cell density of target bio-particles in a sample is illustrated. The conventional biochip includes an interdigitated electrode pair 91, and a surrounding electrode array 92 surrounding the interdigitated electrode pair 91. The interdigitated electrode pair 91 includes a first comb-shaped electrode 911 and a second comb-shaped electrode 912 that are configured to interdigitate with each other and that are spaced apart from each other.

### SUMMARY

Therefore, an object of the disclosure is to provide a biochip for measuring a cell density of target bio-particles in a sample that can alleviate at least one of the drawbacks of the prior art.

According to the disclosure, the biochip includes a spiral electrode pair and a concentric electrode array.

The spiral electrode pair includes a first spiral electrode and a second spiral electrode that are parallel to each other and that are spaced apart from each other. Each of the first spiral electrode and the second spiral electrode is shaped in a spiral extending in one of clockwise and counterclockwise directions.

The concentric electrode array includes a plurality of arcs that are concentric and extend in a circular direction around the spiral electrode pair.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a fragmentary schematic diagram illustrating a conventional biochip for measuring a cell density of target bio-particles in a sample.

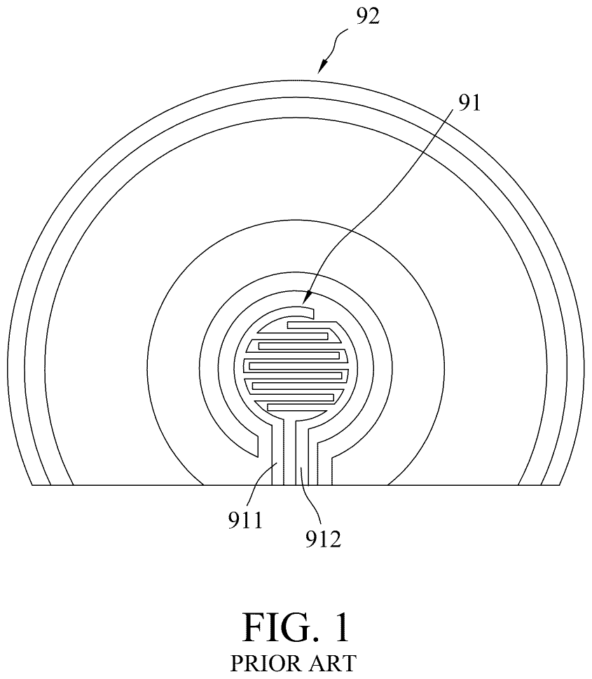

FIG. 2 is a schematic diagram illustrating a biochip for measuring a cell density of target bio-particles in a sample according to an embodiment of the disclosure.

FIG. 3 is a schematic diagram illustrating the biochip operating in a pre-sensing mode.

FIG. 4 is a schematic diagram illustrating the biochip operating in a sensing mode.

FIG. 5 shows schematic diagrams illustrating an impedance model for a sample that contains only buffer.

FIG. 6 shows schematic diagrams illustrating an impedance model for a sample that contains buffer and target bio-particles, where the target bio-particles are evenly distributed in the buffer.

FIG. 7 shows schematic diagrams illustrating an impedance model for a sample that contains buffer and target bio-particles, wherein the target bio-particles are concentrated by the biochip around a spiral electrode pair of the biochip.

FIG. 8 is a schematic diagram illustrating microscopic images of the biochip that was placed in four samples respectively having four concentrations (i.e., 105 CFU/ml, 10⁶ CFU/ml, 107 CFU/ml and 10⁸ CFU/ml) of Escherichia coli (E. coli) and that operated in the pre-sensing mode for six minutes.

FIG. 9 is a plot illustrating a change rate of impedance measured by the biochip against a concentration of E. coli in a sample.

FIG. 10 is a plot illustrating a change rate of impedance measured by the biochip against a concentration of target bio-particles in a sample, wherein six lines respectively represent six kinds of target bio-particles.

FIG. 11 is a schematic diagram illustrating a spiral electrode pair of the biochip according to an embodiment of the disclosure.

### DETAILED DESCRIPTION

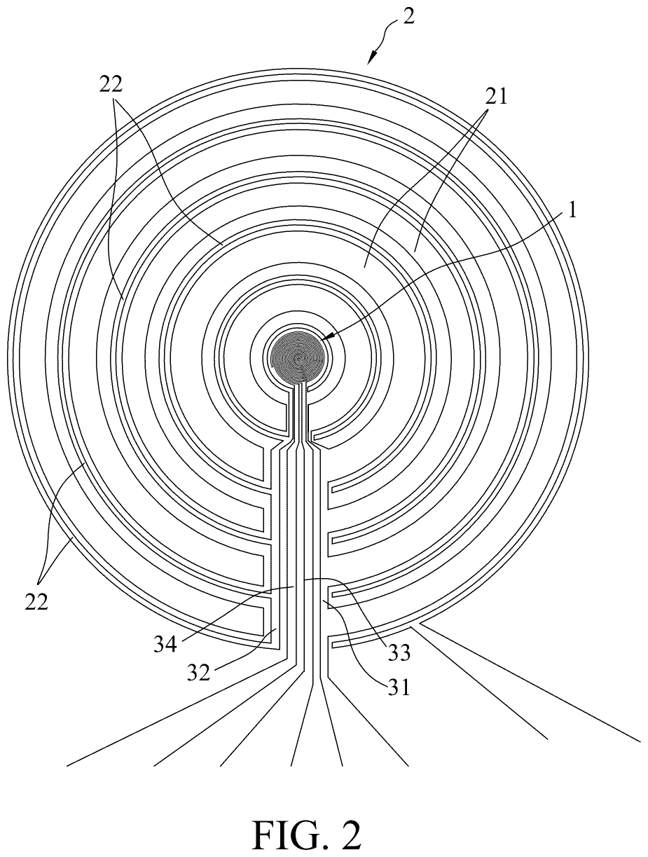

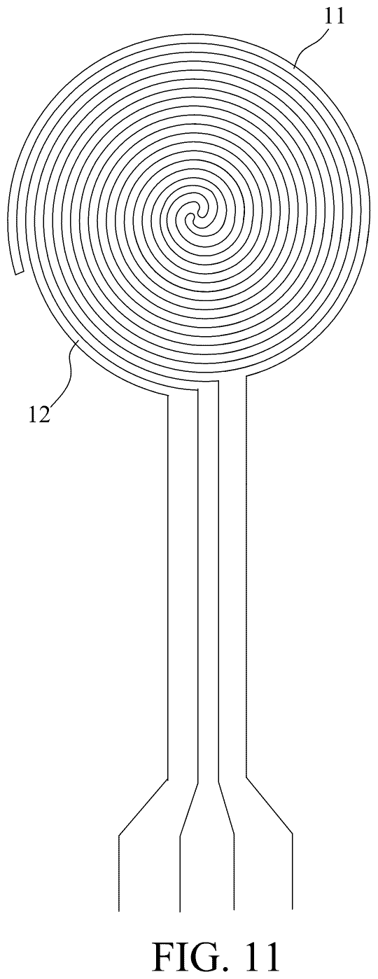

Referring to FIGS. 2 and 11, an embodiment of a biochip for measuring a cell density (also referred to as a concentration) of target bio-particles in a sample according to the disclosure is illustrated. The target bio-particles are exemplarily bacteria, but are not limited thereto. The sample is exemplarily a blood sample, but is not limited thereto.

The biochip includes a spiral electrode pair 1 and a concentric electrode array 2.

The spiral electrode pair 1 includes a first spiral electrode 11 and a second spiral electrode 12 that are parallel to each other and that are spaced apart from each other. Each of the first spiral electrode 11 and the second spiral electrode 12 is shaped in a spiral extending in one of clockwise and counterclockwise directions. In this embodiment, each of the first spiral electrode 11 and the second spiral electrode 12 is exemplarily shaped in a spiral extending in the counterclockwise direction.

More specifically, the first spiral electrode 11 is made of a first conductive strip that is substantially shaped in a spiral and that has a first width, and the second spiral electrode 12 is made of a second conductive strip that is substantially shaped in a spiral and that has a second width identical to the first width. The first conductive strip and the second conductive strip are arranged as two intertwined spirals.

The first spiral electrode 11 has a central end close to a center of the spiral, and an exterior end extending away from the central end in the one of clockwise and counterclockwise directions. In this embodiment, the exterior end of the first spiral electrode 11 exemplarily extends away from the central end of the first spiral electrode 11 in the counterclockwise direction.

Similarly, the second spiral electrode 12 has a central end close to a center of the spiral, and an exterior end extending away from the central end in the one of clockwise and counterclockwise directions. In this embodiment, the exterior end of the second spiral electrode 12 exemplarily extends away from the central end of the second spiral electrode 12 in the counterclockwise direction.

The concentric electrode array 2 includes a plurality of arcs that are concentric and extend in a circular direction around the spiral electrode pair 1.

Specifically, the arcs of the concentric electrode array 2 includes a plurality of first arcs 21, each of which is made of a first conductive arc-shaped strip that has a first width, and a plurality of second arcs 22, each of which is made of a second conductive arc-shaped strip that has a second width. The first width of each of the first conductive arc-shaped strips is greater than the second width of each of the second conductive arc-shaped strips.

The first arcs 21 are spaced apart from each other at regular intervals, and the second arcs 22 are spaced apart from each other at regular intervals. The first arcs 21 and the second arcs 22 are arranged alternately in a radial direction.

Each of the first arcs 21 has a connected end and a free end that are opposite to each other in the circular direction. The connected ends respectively of the first arcs 21 are connected to each other. The free ends respectively of the first arcs 21 are aligned with and spaced apart from each other in the radial direction.

Similarly, each of the second arcs 22 has a connected end and a free end that are opposite to each other in the circular direction. The connected ends respectively of the second arcs 22 are connected to each other. The free ends respectively of the second arcs 22 are aligned with and spaced apart from each other in the radial direction.

The connected ends of the first arcs 21 and the free ends of the second arcs 22 are arranged alternatively. The free ends of the first arcs 21 and the connected ends of the second arcs 22 are arranged alternatively.

The biochip further includes a first receiving line 31, a second receiving line 32, a third receiving line 33 and a fourth receiving line 34. The first receiving line 31, the second receiving line 32, the third receiving line 33 and the fourth receiving line 34 are parallel to and spaced apart from each other, and extend in the radial direction. The connected ends of the first arcs 21 are connected to each other via the first receiving line 31. The connected ends of the second arcs 22 are connected to each other via the second receiving line 32. The exterior end of the first spiral electrode 11 is connected to the third receiving line 33. The exterior end of the second spiral electrode 12 is connected to the fourth receiving line 34.

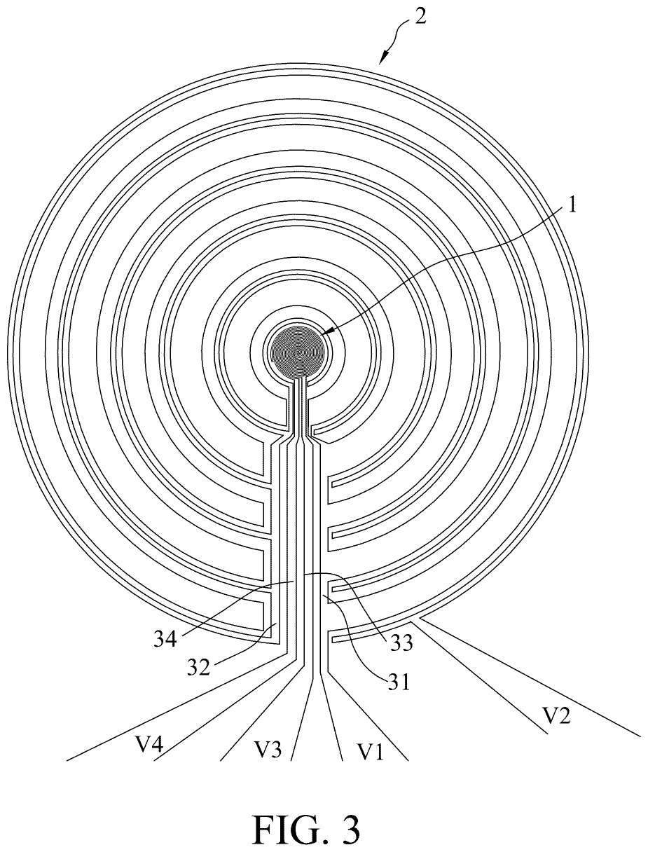

Referring to FIG. 3, the biochip is configured to operate in a pre-sensing mode for concentrating the target bio-particles around the spiral electrode pair 1 when the first receiving line 31 receives a first pre-sensing voltage (V1), the second receiving line 32 receives a second pre-sensing voltage (V2), the third receiving line 33 receives a third pre-sensing voltage (V3) and the fourth receiving line 34 receives a fourth pre-sensing voltage (V4), wherein the first pre-sensing voltage (V1) is greater than the second pre-sensing voltage (V2), the second pre-sensing voltage (V2) is greater than the third pre-sensing voltage (V3), and the third pre-sensing voltage (V3) is not smaller than the fourth pre-sensing voltage (V4) (i.e., V1>V2>V3 V4). In this embodiment, the first pre-sensing voltage (V1) ranges from 5 V to 25 V, the second pre-sensing voltage (V2) ranges from 3 V to 22 V, the third pre-sensing voltage (V3) ranges from 0 V to 20 V, and the fourth pre-sensing voltage (V4) ranges from 0 V to 20 V. Moreover, a difference between the first pre-sensing voltage (V1) and the second pre-sensing voltage (V2) is greater than a difference between the second pre-sensing voltage (V2) and the third pre-sensing voltage (V3), and the difference between the second pre-sensing voltage (V2) and the third pre-sensing voltage (V3) is greater than a difference between the third pre-sensing voltage (V3) and the fourth pre-sensing voltage (V4) (i.e., (V1−V2)>(V2−V3)>(V3−V4)).

It is worth to note that when the biochip operates in the pre-sensing mode, under an electric field generated according to electro-kinetic phenomena of long-range alternating current electro-osmosis (ACEO) and short-range dielectrophoresis (DEP), the target bio-particles would be concentrated around the spiral electrode pair 1 (i.e., a local cell density of the target bio-particles around the spiral electrode pair 1 would be increased) and substances other than the target bio-particles in the sample would be expelled away from the spiral electrode pair 1. In other words, the target bio-particles would be separated from other substances in the sample and would be aggregated around the spiral electrode pair 1. It should be noted that concentrating the target bio-particles around the spiral electrode pair 1 may alleviate exponential decay of a signal strength of an electrical signal that has a Gaussian distribution and that flows away from a surface of the spiral electrode pair 1.

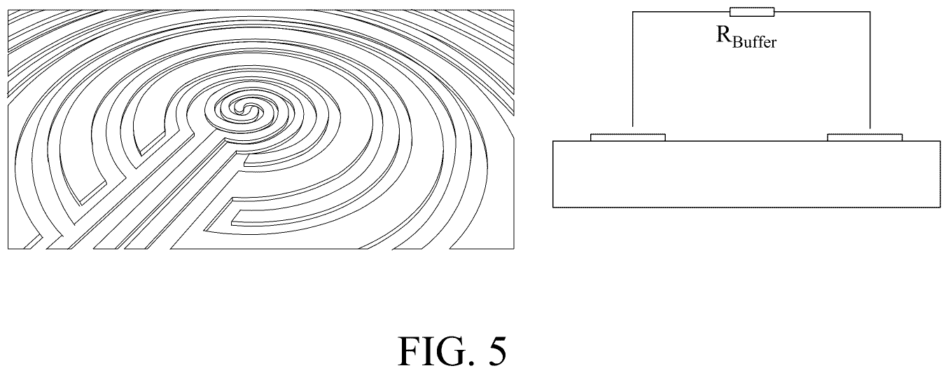

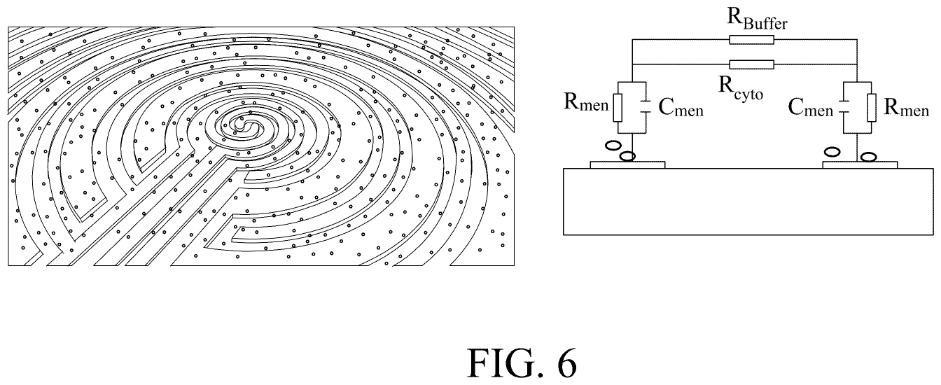

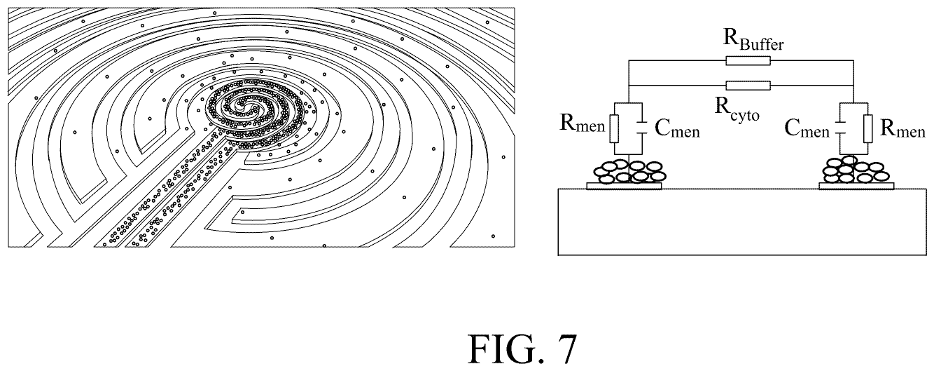

Referring to FIGS. 5 to 7, three impedance models respectively for three different sample conditions are illustrated. In a first sample condition where the sample contains only buffer as shown in FIG. 5, the impedance model for the sample is built as a resistor having buffer resistance (represented by “R_{Buffer}”). In a second sample condition where the sample contains buffer and bacteria (which serve as target bio-particles) and the bacteria are evenly distributed in the buffer as shown in FIG. 6, the impedance model for the sample is built as two RC units respectively connected to two ends of an RR unit in series, wherein each of the two RC units includes a resistor having membrane resistance (represented by “Rₘₑₘ”) and a capacitor having membrane capacitance (represented by “Cₘₑₘ”), the resistor and the capacitor of each of the two RC units are connected in parallel, the RR unit includes the resistor having buffer resistance and a resistor having cell resistance (represented by “R_{cyto}”), and the resistors of the RR unit are connected in parallel. In a third sample condition where the sample contains buffer and bacteria (which serve as target bio-particles) and the bacteria are concentrated by the biochip around the spiral electrode pair 1 of the biochip as shown in FIG. 7, the impedance model for the sample is built in a way similar to that of the second sample condition, i.e., the two RC units are respectively connected to the two ends of the RR unit in series.

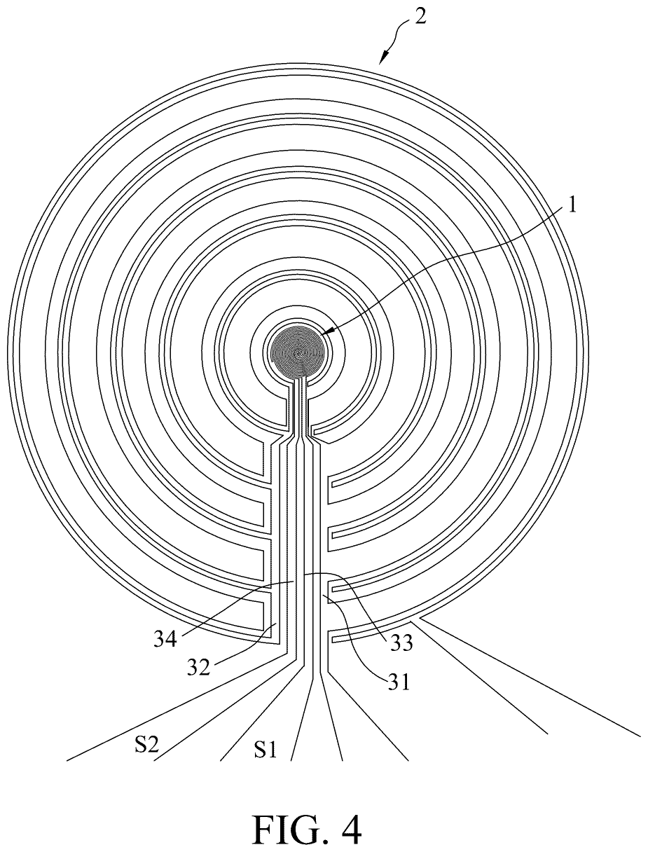

Referring to FIG. 4, the biochip is further configured to operate in a sensing mode for measuring the cell density of target bio-particles in the sample when the first pre-sensing voltage (V1), the second pre-sensing voltage (V2), the third pre-sensing voltage (V3) and the fourth pre-sensing voltage (V4) are all changed to zero and then the third receiving line 33 receives a first sensing sub-signal (S1) and the fourth receiving line 34 receives a second sensing sub-signal (S2). The first sensing sub-signal (S1) and the second sensing sub-signal (S2) cooperatively serve as a sensing signal. Each of the first sensing sub-signal (S1) and the second sensing sub-signal (S2) may have a waveform of one of a sine wave, a square wave and a triangle wave. Each of the first sensing sub-signal (S1) and the second sensing sub-signal (S2) has a peak-to-peak voltage ranging from 0.3 V to 20 V, and has a frequency ranging from 50 Hz to 20 MHz. It should be noted that a time interval between a prior event, where the first pre-sensing voltage (V1), the second pre-sensing voltage (V2), the third pre-sensing voltage (V3) and the fourth pre-sensing voltage (V4) are turned off, and a later event, where the sensing signal (i.e., the first sensing sub-signal (S1) and the second sensing sub-signal (S2)) is applied, is less than one second. Then, impedance of the sample measured by the biochip can be determined based on the sensing signal.

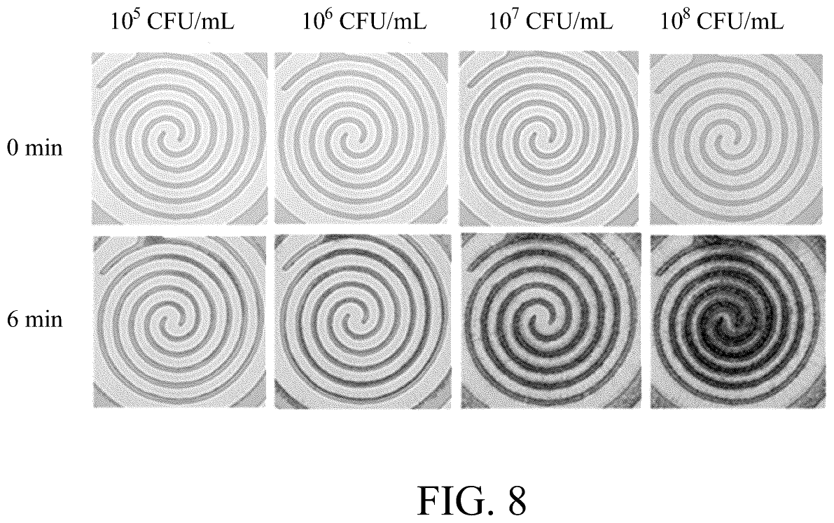

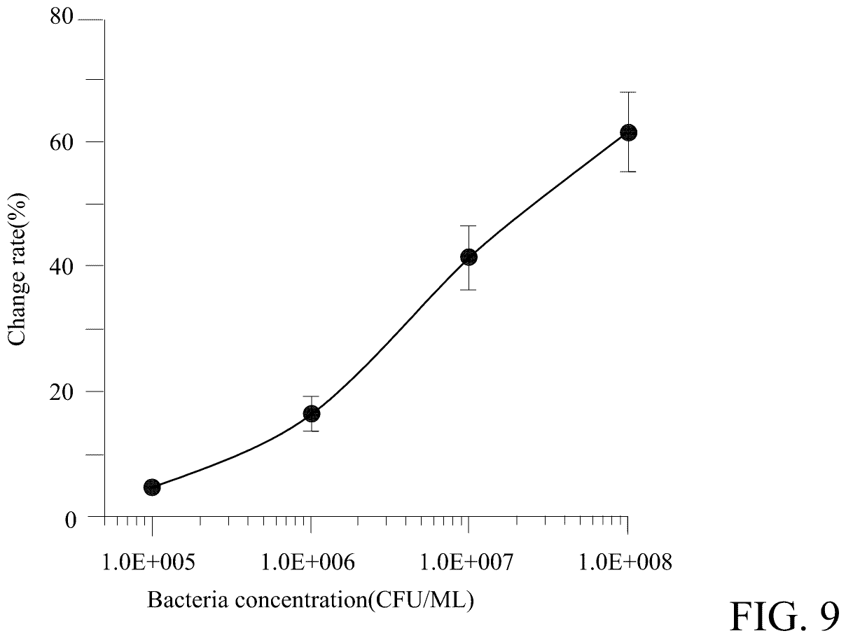

Determination of a change rate of impedance involves comparing measured impedance of a sample that contains only buffer and measured impedance of a sample that contains buffer and a certain concentration of target bio-particles. A change rate of impedance represents a percentage of variation of measured impedance. It is worth to note that a change rate of impedance measured by the biochip is positively correlated with a concentration of target bio-particles in a sample. That is to say, the higher the local cell density of the target bio-particles around the spiral electrode pair 1, the greater the change rate regarding impedance that is measured by the biochip. Referring to an experiment shown in FIG. 8 where Escherichia coli (E. coli) serves as the target bio-particles, the biochip was placed in four samples respectively having four concentrations (i.e., 105 CFU/ml, 10⁶ CFU/ml, 107 CFU/ml and 10⁸ CFU/ml) of E. coli, and the biochip operated in the pre-sensing mode for a six-minute time period. The first pre-sensing voltage (V1) is a peak-to-peak voltage of 12 V, the second pre-sensing voltage (V2) is a peak-to-peak voltage of 7 V, the third pre-sensing voltage (V3) is a peak-to-peak voltage of 3 V, the fourth pre-sensing voltage (V4) is 0 V, and each of the first pre-sensing voltage (V1), the second pre-sensing voltage (V2), the third pre-sensing voltage (V3) and the fourth pre-sensing voltage (V4) is superposed on a direct current (DC) bias voltage of 0.3 V. Four images in an upper row of FIG. 8 were taken at the start of the six-minute time period, and four images in a lower row of FIG. 8 were taken at the end of the six-minute time period. FIG. 9 illustrates a change rate of impedance measured by the biochip against a concentration of E. coli in a sample according to a result of the experiment shown in FIG. 8.

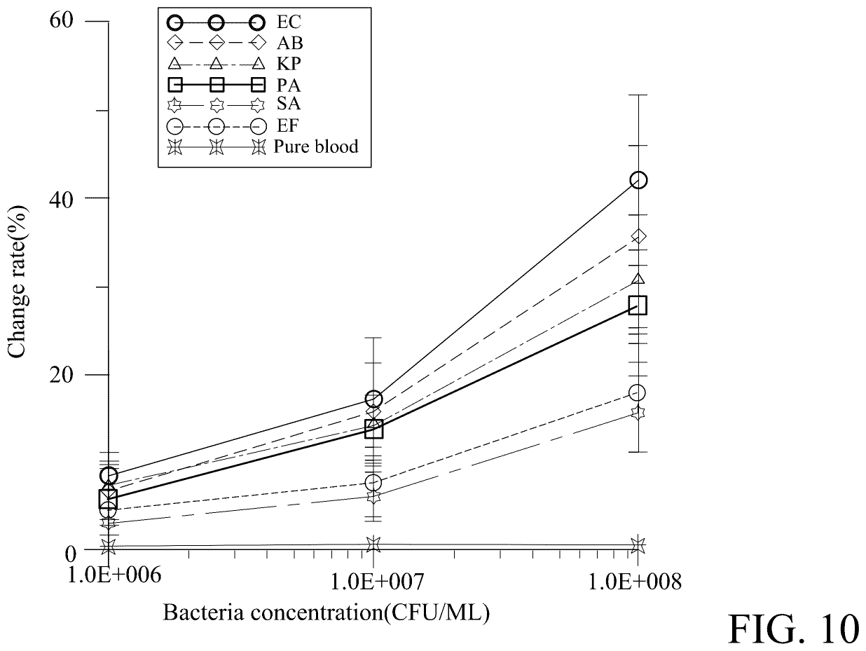

FIG. 10 illustrates a plot of a change rate of impedance measured by the biochip against a concentration of target bio-particles in a sample where the sample includes 50-fold diluted blood, and six septicemia bacteria, i.e., E. coli (represented by “EC”), Klebsiella pneumoniae (K. pneumonia, represented by “KP”), Acinetobacter baumannii (A. baumannii, represented by “AB”), Pseudomonas aeruginosa (P. aeruginosa, represented by “PA”), Staphylococcus aureus (S. aureus, represented by “SA”) and Enterococcus faecium (E. faecium, represented by “EF”), which serve as the target bio-particles. In addition, a pure blood sample serves as a control group for reference. The plot shows a significant positive correlation between the change rate of impedance measured by the biochip and the concentration of target bio-particles. Moreover, a change rate for the control group (i.e., the pure blood sample) is less than 2%, which implies that interference caused by blood cells was alleviated by the biochip of the disclosure (i.e., became less severe).

In a scenario where the target bio-particles are bacteria and other substances in the sample are blood cells, when the biochip operates in the pre-sensing mode, the bacteria would be trapped on a surface of the spiral electrode pair 1 of the biochip because of convergent electric field focus caused by positive dielectrophoretic (pDEP) force, and the blood cells would be expelled away from the spiral electrode pair 1 of the biochip because of a uniform depletion of negative dielectrophoretic (nDEP) force. It is worth to note that interference caused by the blood cells would exponentially decay under the nDEP force. Such bacteria attraction capability enhances a signal strength of the sensing signal and blood-cell depletion capability reduces interference caused by the blood cells, thereby reducing limit of detection (LOD) and improving accuracy of measurement.

To sum up, the biochip for measuring a cell density of target bio-particles in a sample according to the disclosure includes the spiral electrode pair 1 and the concentric electrode array 2. The spiral electrode pair 1 includes the first spiral electrode 11 and the second spiral electrode 12 that are parallel to each other and that are spaced apart from each other, and each of the first spiral electrode 11 and the second spiral electrode 12 is shaped in a spiral. The concentric electrode array 2 includes the arcs that are concentric and extend in a circular direction around the spiral electrode pair 1. By applying different combinations of voltages to the spiral electrode pair 1 and the concentric electrode array 2, the biochip is capable of operating in the pre-sensing mode for concentrating the target bio-particles, and the sensing mode for measuring the cell density of target bio-particles in the sample. Compared with an interdigitated electrode pair that is utilized in a conventional biochip, measurement of a cell density of target bio-particles performed by using the spiral electrode pair 1 of the biochip according to the disclosure may achieve a relatively excellent LOD of 105 CFU/ml and a relatively wide detection range from 105 CFU/ml to 107 CFU/ml. Furthermore, depletion efficiency of the spiral electrode pair 1 of the biochip according to the disclosure is more excellent than that of the interdigitated electrode pair of the conventional biochip.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A biochip for measuring a cell density of target bio-particles in a sample, said biochip comprising:
a spiral electrode pair including a first spiral electrode and a second spiral electrode that are parallel to each other and that are spaced apart from each other, each of said first spiral electrode and said second spiral electrode being shaped in a spiral extending in one of clockwise and counterclockwise directions; and
a concentric electrode array including a plurality of arcs that are concentric and extend in a circular direction around said spiral electrode pair.

2. The biochip as claimed in claim 1, wherein said first spiral electrode is made of a first conductive strip that is substantially shaped in a spiral and that has a first width, and said second spiral electrode is made of a second conductive strip that is substantially shaped in a spiral and that has a second width identical to the first width.

3. The biochip as claimed in claim 1, wherein said first spiral electrode is made of a first conductive strip that is substantially shaped in a spiral, and said second spiral electrode is made of a second conductive strip that is substantially shaped in a spiral, and
wherein said first conductive strip and said second conductive strip are arranged as two intertwined spirals.

4. The biochip as claimed in claim 3, wherein:
said first spiral electrode has a central end close to a center of the spiral, and an exterior end extending away from said central end in the one of clockwise and counterclockwise directions; and
said second spiral electrode has a central end close to a center of the spiral, and an exterior end extending away from said central end in the one of clockwise and counterclockwise directions.

5. The biochip as claimed in claim 4, further comprising a first receiving line and a second receiving line that extend in a radial direction and that are parallel to and spaced apart from each other,
wherein said exterior end of said first spiral electrode is connected to said first receiving line, and said exterior end of said second spiral electrode is connected to said second receiving line.

6. The biochip as claimed in claim 1, wherein said arcs of said concentric electrode array includes a plurality of first arcs, each of which is made of a first conductive arc-shaped strip that has a first width, and a plurality of second arcs, each of which is made of a second conductive arc-shaped strip that has a second width, and the first width of each of said first conductive arc-shaped strips is greater than the second width of each of said second conductive arc-shaped strips.

7. The biochip as claimed in claim 6, wherein said first arcs are spaced apart from each other at regular intervals, and said second arcs are spaced apart from each other at regular intervals.

8. The biochip as claimed in claim 6, wherein said first arcs and said second arcs are arranged alternately in a radial direction.

9. The biochip as claimed in claim 6, wherein:
each of said first arcs has a connected end and a free end that are opposite to each other in the circular direction, said connected ends respectively of said first arcs are connected to each other, and said free ends respectively of said first arcs are aligned with and spaced apart from each other in a radial direction;
each of said second arcs has a connected end and a free end that are opposite to each other in the circular direction, said connected ends respectively of said second arcs are connected to each other, and said free ends respectively of said second arcs are aligned with and spaced apart from each other in the radial direction; and
said connected ends of said first arcs and said free ends of said second arcs are arranged alternatively, and said free ends of said first arcs and said connected ends of said second arcs are arranged alternatively.

10. The biochip as claimed in claim 9, further comprising a first receiving line and a second receiving line that extend in the radial direction and that are parallel to and spaced apart from each other,
wherein said connected ends of said first arcs are connected to each other via said first receiving line, and said connected ends of said second arcs are connected to each other via said second receiving line.

11. The biochip as claimed in claim 1, wherein:
said first spiral electrode is made of a first conductive strip that is substantially shaped in a spiral, and said second spiral electrode is made of a second conductive strip that is substantially shaped in a spiral;
said first conductive strip and said second conductive strip are arranged as two intertwined spirals;
said arcs of said concentric electrode array include a plurality of first arcs and a plurality of second arcs; and
said first arcs and said second arcs are arranged alternately in a radial direction.

12. The biochip as claimed in claim 11, wherein:
said first spiral electrode has a central end close to a center of the spiral, and an exterior end extending away from said central end in the one of clockwise and counterclockwise directions;
said second spiral electrode has a central end close to a center of the spiral, and an exterior end extending away from said central end in the one of clockwise and counterclockwise directions;
each of said first arcs has a connected end and a free end that are opposite to each other in the circular direction, said connected ends respectively of said first arcs are connected to each other, and said free ends respectively of said first arcs are aligned with and spaced apart from each other in the radial direction;
each of said second arcs has a connected end and a free end that are opposite to each other in the circular direction, said connected ends respectively of said second arcs are connected to each other, and said free ends respectively of said second arcs are aligned with and spaced apart from each other in the radial direction;
said connected ends of said first arcs and said free ends of said second arcs are arranged alternatively, and said free ends of said first arcs and said connected ends of said second arcs are arranged alternatively;
said biochip further comprises a first receiving line, a second receiving line, a third receiving line and a fourth receiving line parallel to and spaced apart from each other and extending in the radial direction;
said connected ends of said first arcs are connected to each other via said first receiving line, and said connected ends of said second arcs are connected to each other via said second receiving line;
said exterior end of said first spiral electrode is connected to said third receiving line, and said exterior end of said second spiral electrode is connected to said fourth receiving line; and
said biochip is configured to operate in a pre-sensing mode for concentrating the target bio-particles around said spiral electrode pair when said first receiving line receives a first pre-sensing voltage, said second receiving line receives a second pre-sensing voltage, said third receiving line receives a third pre-sensing voltage and said fourth receiving line receives a fourth pre-sensing voltage, the first pre-sensing voltage being greater than the second pre-sensing voltage, the second pre-sensing voltage being greater than the third pre-sensing voltage, the third pre-sensing voltage being not smaller than the fourth pre-sensing voltage.

13. The biochip as claimed in claim 12, wherein said biochip is further configured to operate in a sensing mode for measuring the cell density of target bio-particles in the sample when the first pre-sensing voltage, the second pre-sensing voltage, the third pre-sensing voltage and the fourth pre-sensing voltage are all changed to zero and then said third receiving line receives a first sensing sub-signal and said fourth receiving line receives a second sensing sub-signal.

14. The biochip as claimed in claim 1, wherein:
said first spiral electrode is made of a first conductive strip that is substantially shaped in a spiral and that has a first width, and said second spiral electrode is made of a second conductive strip that is substantially shaped in a spiral and that has a second width identical to the first width;
said first conductive strip and said second conductive strip are arranged as two intertwined spirals;
said first spiral electrode has a central end close to a center of the spiral, and an exterior end extending away from said central end in the one of clockwise and counterclockwise directions;
said second spiral electrode has a central end close to a center of the spiral, and an exterior end extending away from said central end in the one of clockwise and counterclockwise directions;
said arcs of said concentric electrode array include a plurality of first arcs, each of which is made of a first conductive arc-shaped strip that has a first width, and a plurality of second arcs, each of which is made of a second conductive arc-shaped strip that has a second width, the first width of each of said first conductive arc-shaped strips being greater than the second width of each of said second conductive arc-shaped strips;
said first arcs are spaced apart from each other at regular intervals, and said second arcs are spaced apart from each other at regular intervals;
said first arcs and said second arcs are arranged alternately in a radial direction;
each of said first arcs has a connected end and a free end that are opposite to each other in the circular direction, said connected ends respectively of said first arcs are connected to each other, and said free ends respectively of said first arcs are aligned with and spaced apart from each other in the radial direction;
each of said second arcs has a connected end and a free end that are opposite to each other in the circular direction, said connected ends respectively of said second arcs are connected to each other, and said free ends respectively of said second arcs are aligned with and spaced apart from each other in the radial direction; and
said connected ends of said first arcs and said free ends of said second arcs are arranged alternatively, and said free ends of said first arcs and said connected ends of said second arcs are arranged alternatively.

15. The biochip as claimed in claim 14, further comprising a first receiving line, a second receiving line, a third receiving line and a fourth receiving line parallel to and spaced apart from each other and extending in the radial direction, wherein:
said connected ends of said first arcs are connected to each other via said first receiving line, and said connected ends of said second arcs are connected to each other via said second receiving line;
said exterior end of said first spiral electrode is connected to said third receiving line, and said exterior end of said second spiral electrode is connected to said fourth receiving line; and
said biochip is configured to operate in a pre-sensing mode for concentrating the target bio-particles around said spiral electrode pair when said first receiving line receives a first pre-sensing voltage, said second receiving line receives a second pre-sensing voltage, said third receiving line receives a third pre-sensing voltage and said fourth receiving line receives a fourth pre-sensing voltage, the first pre-sensing voltage being greater than the second pre-sensing voltage, the second pre-sensing voltage being greater than the third pre-sensing voltage, the third pre-sensing voltage being not smaller than the fourth pre-sensing voltage.

16. The biochip as claimed in claim 15, wherein said biochip is further configured to operate in a sensing mode for measuring the cell density of target bio-particles in the sample when the first pre-sensing voltage, the second pre-sensing voltage, the third pre-sensing voltage and the fourth pre-sensing voltage are all changed to zero and then said third receiving line receives a first sensing sub-signal and said fourth receiving line receives a second sensing sub-signal.

17. The biochip as claimed in claim 1, wherein the target bio-particles are bacteria.

18. The biochip as claimed in claim 1, wherein the sample is a blood sample.
